(19) United States
(12) Patent Application Publication
(10) Pub. No.: US 2021/0082552 A1
(43) Pub. Date: Mar. 18, 2021

(54) SYSTEMS AND METHODS FOR PREDICTING SKIN TREATMENT OUTCOMES USING SKIN PH INFORMATION

(71) Applicant: L'Oreal, Paris (FR)

(72) Inventors: Rafal Michal Pielak, El Cerrito, CA (US); Casey Barbarino, San Francisco, CA (US)

(73) Assignee: L'Oreal, Paris (US)

(21) Appl. No.: 16/572,401

(22) Filed: Sep. 16, 2019

(51) Int. Cl. G16H 15/00; G16H 30/20; G16H 20/10

(52) U.S. Cl. CPC G16H 15/00; G16H 20/10; G16H 30/20

(57) ABSTRACT

An electronic sensor for pH measurement may be used (e.g. an ISFET sensor, a glass electrode sensor, an antimony electrode sensor) to collect skin pH information from a subject. An application is provided on a computing device that detects and analyzes the pH measurements reported by the electronic sensor to determine skin pH values for analysis. Based on the determined skin pH values and questionnaire responses, the application may recommend a skin regimen/treatment to restore skin function. The application may also provide visualizations of predicted effects of the skin regimen on a skin condition of the subject.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In some embodiments, a system for generating predictive visualizations of a result of a skin treatment regimen is provided. The system comprises a server computing system, at least one electronic measurement device, and a mobile computing device. The server computing system comprises one or more computing devices. The at least one electronic measurement device is configured to produce measurements of a skin area of a subject on which the electronic measurement device is placed. The mobile computing device comprises at least one processor, a display device, a camera, and a non-transitory computer-readable medium having computer-executable instructions stored thereon. The instructions, in response to execution by the at least one processor, cause the mobile computing device to capture an image of an area of interest of the skin of the subject, receive measurements from the electronic measurement device, determine a pH value representing a pH of the skin area of the subject by analyzing the measurements produced by the electronic measurement device, and transmit the pH value and the image of the area of interest to the server computing system. The server computing system is configured to determine a skincare product recommendation based on the pH value; generate a visualization indicating a result of applying the skincare product to the area of interest based on the pH value, the image of the area of interest, and the skincare product; and transmit the visualization to the mobile computing device. The instructions also cause the mobile computing device to receive and present the visualization to the subject.

In some embodiments, a method for treating a patient with a skin condition associated with high skin pH is provided. A skin pH of skin of the patient is determined by obtaining at least one measurement using at least one electronic measurement device from the skin of the patient, wherein the at least one measurement includes an electrical potential of the skin; and using a mobile computing device to determine the skin pH based on the electrical potential of the skin. A recommended skincare product to treat the skin condition is determined based on the skin pH. The recommended skincare product is topically administered to the skin.

In some embodiments, a method of generating a rendering of a face image to indicate a result of a skin treatment regimen is provided. A mobile computing device receives a measurement of a skin area of a subject, wherein the measurement is associated with a pH of the skin area. The mobile computing device captures an image of a face of the subject. The mobile computing device determines a pH value based on the measurement. The mobile computing device determines a recommended skincare product based on the pH value. The mobile computing device determines a predicted face image based on the pH value, the recommended skincare product, and the second image. The mobile computing device presents the predicted face image to the subject.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic diagram that illustrates a non-limiting example embodiment of a system according to various aspects of the present disclosure;

FIG. 2 is a block diagram that illustrates various components of a non-limiting example embodiment of a mobile computing device and a non-limiting example embodiment of a server computing system according to various aspects of the present disclosure;

FIGS. 3A and 3B are illustrations showing an example of a first image taken of a subject and a predictive visualization generated of the subject according to various aspects of the present disclosure;

FIGS. 4A-4C are a flowchart that illustrates a non-limiting example embodiment of a method of generating a predictive visualization of a subject's response to a recommended skincare regimen according to various aspects of the present disclosure;

FIG. 5 is a flowchart that illustrates a non-limiting example embodiment of a method of determining pH values based on measurements generated by an electronic measurement device according to various aspects of the present disclosure;

FIG. 6 is a flowchart that illustrates a non-limiting example embodiment of a method of determining pH values based on measurements generated by an electronic measurement device using machine learning according to various aspects of the present disclosure;

FIG. 7A is a block diagram that illustrates example components included within a non-limiting example embodiment of an electronic measurement device 106 according to various aspects of the present disclosure;

FIG. 7B illustrates a non-limiting example embodiment of an electronic measurement device according to various aspects of the present disclosure; and

FIG. 7C illustrates a non-limiting example embodiment of a measurement surface of an electronic measurement device according to various aspects of the present disclosure.

### DETAILED DESCRIPTION

Skin pH is a vital component of the normal function of human skin. Dysregulation of the acidity plays a role in a number of diseases, such as atopic dermatitis, eczema, and acne vulgaris. As some non-limiting examples: pH is typically found to be significantly higher in eczematous skin. Higher pH values have been measured in areas corresponding to more intense itching and skin dryness in atopic dermatitis (AD). Free amino acids and urocanic acid, which are involved in creating the acidic milieu of the stratum corneum (SC), are markedly reduced in AD. Filaggrin, a protein precursor of free amino acids, is deficient in AD. Sweat secretion, which is rich in lactic acid that contributes to the acid mantle, is reduced in AD. Impaired barrier function in AD can be explained in part by disturbed synthesis, excretion, and maturation of SC lipids, process that depends on enzymes with acidic pH optima. Aberrant lipid organization, namely increased gel phase relative to the crystalline phase of lamellar structures, has been described in patients with AD. Lamellar liquid crystal formation occurs at pH values of 4.5-6. Colonization with S. aureus is a common feature of patients with AD and is considered a major pathogenic factor in AD. Growth of Staphylococcal strains is maximal at neutral pH and markedly inhibited at pH values around 5. In vitro, P. acnes grows well at pH values between 6 and 6.5 and growth is reduced at pH values less than 6. In a study of acne-prone patients, the number of facial inflammatory lesions was compared in subjects using a conventional alkaline soap versus those using an acidic syndet bar.

Skin care products can either exacerbate skin conditions or ameliorate them. Exposure to exogenous agents such as cleansers, creams, deodorants, and topical antibacterials affect pH and can further exacerbate underlying disease. Selection of topical agents that preserve an acidic environment seems relevant in these patients. Non-soap-based surfactants are known as syndets (synthetic detergent-based bars or liquids). Syndets are generally neutral or acidic, while soap-based cleansers are alkaline. Topical alpha-hydroxy acids (AHA) are common agents used in treating disorders of keratinization. AHA, such as lactic acid, has been shown to increase ceramide production by human keratinocytes by 300% in vitro. Twice daily application of 4% l-lactic acid formulations (pH 3.7-4.0) led to significant improvements in barrier function. Studies have shown beneficial effects of topical acidic electrolyte water (pH 2.0-2.7) on the severity of dermatitis and S. aureus colonization of the skin.

Despite knowledge of these characteristics of how skin conditions react to skin pH, there is not currently any convenient way to collect information regarding pH associated with skin and to use such information to recommend treatment regimens for the skin conditions. What is desired are devices and methods that allow analysis of pH to be used to recommend skincare products.

In some embodiments of the present disclosure, an electronic sensor for pH measurement may be used (e.g. an ISFET sensor, a glass electrode sensor, an antimony electrode sensor) to collect skin pH information from a subject. An application is provided on a computing device that detects and analyzes the pH measurements reported by the electronic sensor to determine skin pH values for analysis. Based on the determined skin pH values and questionnaire responses, the application may recommend a skin regimen/treatment to restore skin function.

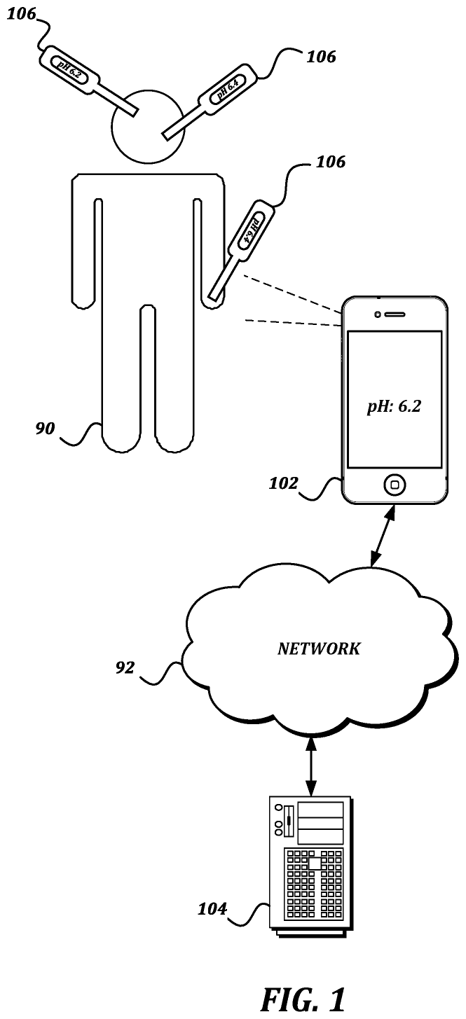

FIG. 1 is a schematic diagram that illustrates a non-limiting example embodiment of a system according to various aspects of the present disclosure. In the illustrated system, the skin pH of a subject 90 is measured using one or more electronic measurement devices 106. As shown, four electronic measurement devices 106 are used to determine pH measurements for various skin regions of the subject 90. In some embodiments, multiple different skin regions may be tested in order to compare the results of those regions to each other, or to build a map of various skin pH values. In some embodiments, a single electronic measurement device 106 may be used, and may be moved to each skin region to obtain pH measurements for the different skin regions.

In some embodiments, the electronic measurement device 106 may transmit its pH measurements over a wired or wireless communication medium. A mobile computing device 102 may receive the pH measurements via the communication medium, and may process the pH measurements to determine one or more skin pH values. In some embodiments, the mobile computing device 102 transmits at least the determined skin pH values to a server computing device 104 via a network 92, and the server computing device 104 may respond with a skincare product recommendation to be presented to the subject 90 by the mobile computing device 102. The network 92 may include any suitable wireless communication technology (including but not limited to Wi-Fi, WiMAX, Bluetooth, 2G, 3G, 4G, 5G, and LTE), wired communication technology (including but not limited to Ethernet, USB, and FireWire), or combinations thereof.

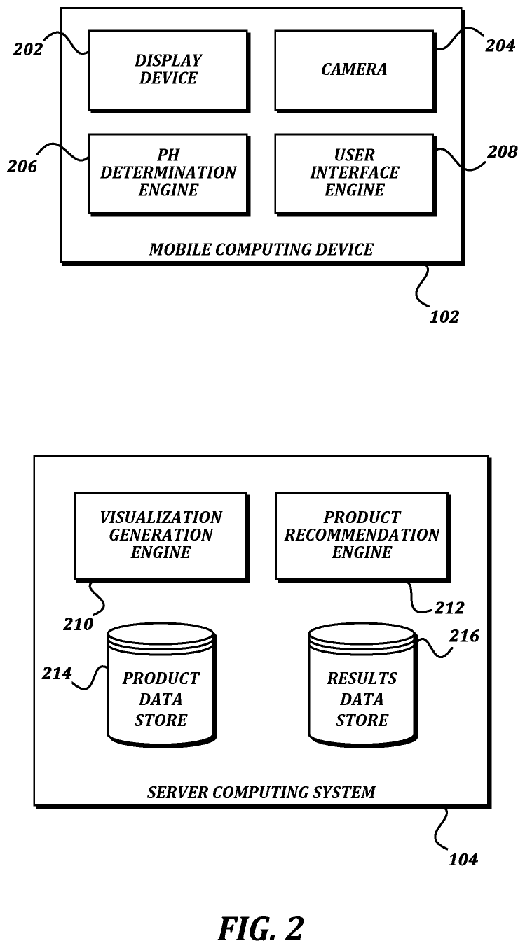

FIG. 2 is a block diagram that illustrates various components of a non-limiting example embodiment of a mobile computing device and a non-limiting example embodiment of a server computing system according to various aspects of the present disclosure. The mobile computing device 102 is configured to collect information from a subject 90 that reflects the skin pH of the subject 90 and/or effectiveness of various skincare products. The mobile computing device 102 then transmits the collected information to the server computing system 104 via the network 92. In some embodiments, the server computing system 104 uses the information to determine a recommendation for a skincare product to be used by the subject 90, and transmits the recommendation back to the mobile computing device 102 to be presented to the subject 90. In some embodiments, the server computing system 104 uses the information to generate visualizations of the subject 90 to show a prediction of how a recommended skincare product would affect a skin condition, and transmits the visualizations back to the mobile computing device 102 to be presented to the subject.

In some embodiments, the mobile computing device 102 may be a smartphone. In some embodiments, the mobile computing device 102 may be any other type of computing device having the illustrated components, including but not limited to a tablet computing device or a laptop computing device. In some embodiments, the mobile computing device 102 may not be mobile, but may instead be a stationary computing device such as a desktop computing device. In some embodiments, the illustrated components of the mobile computing device 102 may be within a single housing. In some embodiments, the illustrated components of the mobile computing device 102 may be in separate housings that are communicatively coupled through wired or wireless connections (such as a laptop computing device with an external camera connected via a USB cable). The mobile computing device 102 also includes other components that are not illustrated, including but not limited to one or more processors, a non-transitory computer-readable medium, a power source, and one or more network communication interfaces.

As shown, the mobile computing device 102 includes a display device 202, a camera 204, a pH determination engine 206, and a user interface engine 208. In some embodiments, the display device 202 is any suitable type of display device, including but not limited to an LED display, an OLED display, or an LCD display, that is capable of presenting interfaces to the subject 90. In some embodiments, the display device 202 may include an integrated touch-sensitive portion that accepts input from the subject 90. In some embodiments, the camera 204 is any suitable type of digital camera that is used by the mobile computing device 102. In some embodiments, the mobile computing device 102 may include more than one camera 204, such as a front-facing camera and a rear-facing camera.

In some embodiments, the pH determination engine 206 is configured to collect information from the electronic measurement devices 106 and to determine skin pH values based on the information. For example, the pH determination engine 206 may receive wireless signals from the electronic measurement devices 106, and may then analyze the wireless signals to determine the skin pH based on the measurements made by the electronic measurement devices 106.

In some embodiments, the user interface engine 208 may be configured to present one or more questionnaires to the subject 90 in order to collect information that may be relevant to the effectiveness of a given skincare product, or to likely environmental effects on a skincare condition. In some embodiments, the user interface engine 208 may be configured to use the camera 204 to capture images of the subject 90, and to present visualizations of the subject 90 received from the server computing system 104.

In some embodiments, the server computing system 104 includes one or more computing devices that each include one or more processors, non-transitory computer-readable media, and network communication interfaces that are collectively configured to provide the components illustrated below. In some embodiments, the one or more computing devices that make up the server computing system 104 may be rack-mount computing devices, desktop computing devices, or computing devices of a cloud computing service.

As shown, the server computing system 104 includes a visualization generation engine 210, a product recommendation engine 212, a product data store 214, and a results data store 216. In some embodiments, the visualization generation engine 210 receives the skin pH information and an image of the subject 90 from the mobile computing device 102, and uses this information to generate visualizations of the subject 90 over time. The visualizations may include predictions of how a skin condition experienced by the subject 90 will change over time. The predictions may be affected by a recommended skincare product, and/or by questionnaire responses provided by the mobile computing device 102. The visualizations may be transmitted to the mobile computing device 102 to be presented to the subject 90.

In some embodiments, the product recommendation engine 212 receives the skin pH information and/or the questionnaire information from the mobile computing device 102, and uses the information to determine an appropriate product stored in a product data store 214 that can address a skin condition experienced by the subject 90. In some embodiments, the product recommendation engine 212 provides the recommended products to the mobile computing device 102 to be presented to the subject 90. In some embodiments, the product recommendation engine 212 may also receive feedback from the subject 90 after having used the recommended products, and may store the feedback in a results data store 216 in order to improve future product recommendations.

In general, the word “engine,” as used herein, refers to logic embodied in hardware or software instructions, which can be written in a programming language, such as C, C++, COBOL, JAVA™, PHP, Perl, HTML, CSS, JavaScript, VBScript, ASPX, Microsoft .NET™, Go, and/or the like. An engine may be compiled into executable programs or written in interpreted programming languages. Software engines may be callable from other engines or from themselves. Generally, the engines described herein refer to logical modules that can be merged with other engines, or can be divided into sub-engines. The engines can be stored in any type of computer-readable medium or computer storage device and be stored on and executed by one or more general purpose computers, thus creating a special purpose computer configured to provide the engine or the functionality thereof.

As understood by one of ordinary skill in the art, a “data store” as described herein may be any suitable device configured to store data for access by a computing device. One example of a data store is a highly reliable, high-speed relational database management system (DBMS) executing on one or more computing devices and accessible over a high-speed network. Another example of a data store is a key-value store. However, any other suitable storage technique and/or device capable of quickly and reliably providing the stored data in response to queries may be used, and the computing device may be accessible locally instead of over a network, or may be provided as a cloud-based service. A data store may also include data stored in an organized manner on a computer-readable storage medium, such as a hard disk drive, a flash memory, RAM, ROM, or any other type of computer-readable storage medium. One of ordinary skill in the art will recognize that separate data stores described herein may be combined into a single data store, and/or a single data store described herein may be separated into multiple data stores, without departing from the scope of the present disclosure.

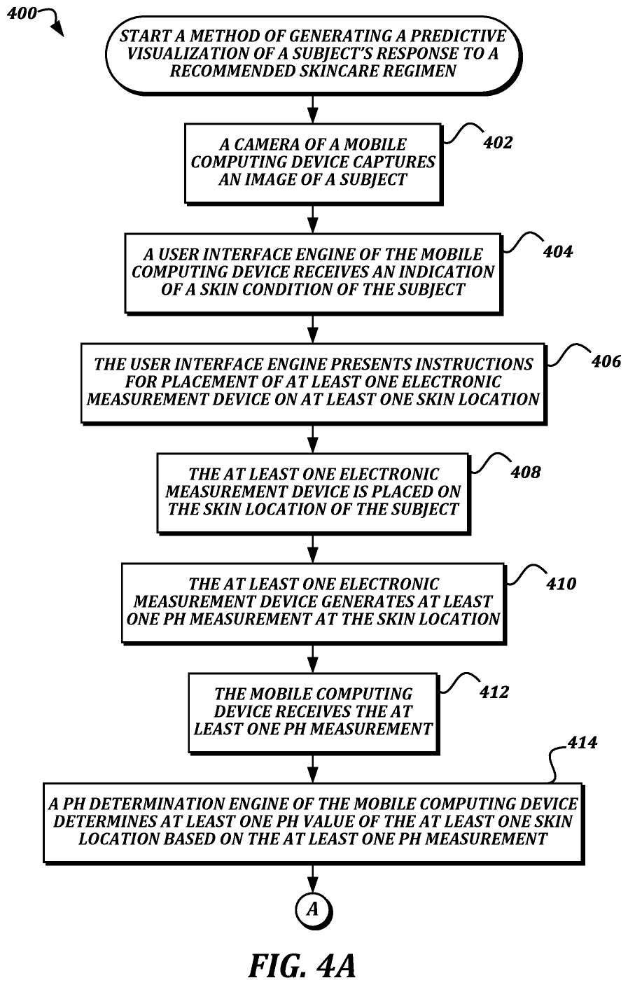

FIGS. 4A-4C are a flowchart that illustrates a non-limiting example embodiment of a method of generating a predictive visualization of a subject's response to a recommended skincare regimen according to various aspects of the present disclosure. In the method 400, the sweat pH of a subject 90 is determined, and an image of the subject is collected. Information regarding the environment of the subject 90 is also collected. A product recommendation is determined, and a visualization is generated to show an expected effect on a skin condition experienced by the subject 90 as a result of using the recommended skincare product.

From a start block, the method 400 proceeds to block 402, where a camera 204 of a mobile computing device 102 captures an image of a subject 90. In some embodiments, the front-facing camera 204 may be used to capture the image of the subject 90, such as a camera 204 that may be used in a “selfie” mode. In other embodiments, a rear-facing camera 204 may be used, particularly in embodiments where an operator other than the subject 90 is operating the mobile computing device 102. At block 404, a user interface engine 208 of the mobile computing device 102 receives an indication of a skin condition of the subject 90. The user interface engine 208 may present a list of conditions, and the subject 90 may select which of the conditions are being experienced (e.g., acne vulgaris, eczema, atopic dermatitis, etc.) from the presented list.

At block 406, the user interface engine 208 presents instructions for placement of at least one electronic measurement device 106 on at least one skin location. In some embodiments, the instructions may include an image that illustrates where the electronic measurement device 106 should be placed (e.g., on the forehead, on the cheekbone, on the nose, on the back of the hand, on the top of the foot) in order to collect skin pH information from a desired area. At block 408, the at least one electronic measurement device 106 is placed on the skin location of the subject 90 in accordance with the instructions. At block 410, the at least one electronic measurement device 106 generates at least one pH measurement at the skin location, and at block 412, the mobile computing device 102 receives the at least one pH measurement. In some embodiments, the pH measurement may include a measurement of a detected pH value, and may also include other measurements, including but not limited to a temperature measurement.

In some embodiments, the user interface engine 208 may present an instruction for placement of the electronic measurement device 106 in a particular location, the electronic measurement device 106 may be placed in the location and may generate at least one pH measurement at the location, and the mobile computing device 102 may receive the at least one pH measurement before the user interface engine 208 presents an instruction to place the electronic measurement device 106 in a subsequent location. In this way, a single electronic measurement device 106 may be used to obtain skin pH measurements from multiple locations on the subject 90.

In some embodiments, more than one pH measurement may be obtained by the electronic measurement device 106 over time from a given skin location. For example, in some embodiments, the electronic measurement device 106 may be a wearable device that is held in place in the skin location for hours, days, weeks, or any other period of time. The wearable device may periodically measure the skin location over which the wearable device is worn, and may store the pH measurements for eventual transmission to the mobile computing device 102.

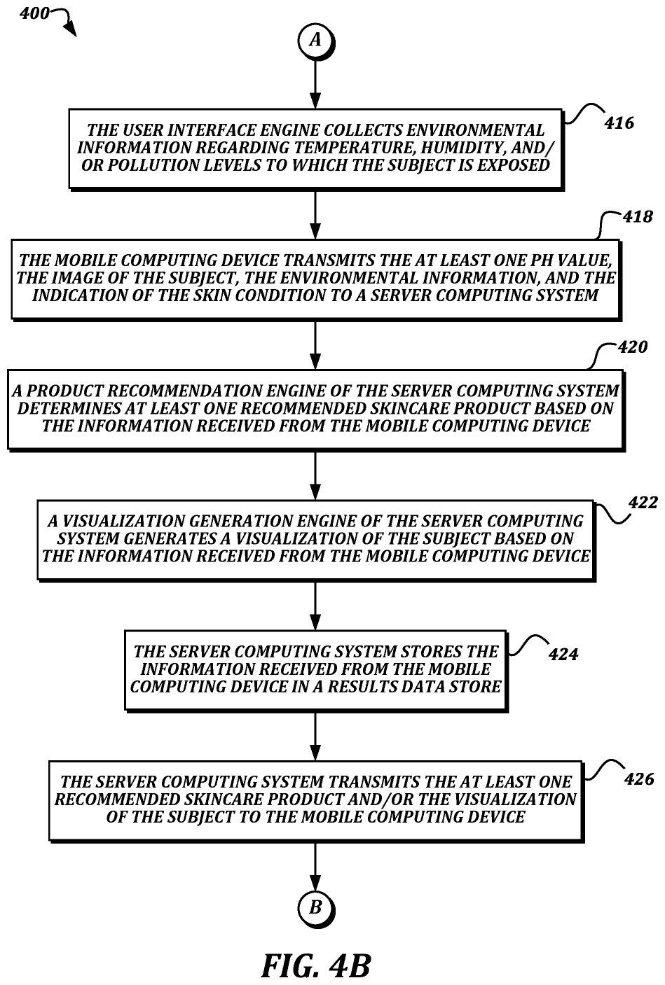

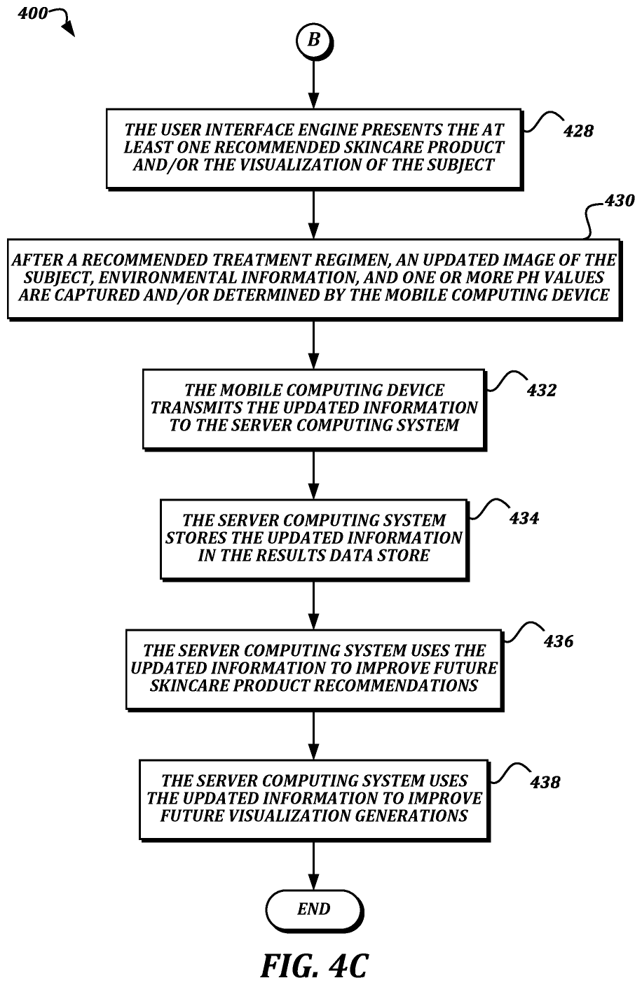

At block 414, a pH determination engine 206 of the mobile computing device 102 determines at least one pH value of the at least one skin location based on the at least one pH measurement. In some embodiments, the pH determination engine 206 may combine multiple pH measurements and/or other information included with pH measurements in order to determine skin pH values of the skin locations. Further description of methods for determining a pH value based on the at least one pH measurement are illustrated in FIGS. 5-6 and are described in detail below.

The method 400 then proceeds to a continuation terminal (“terminal A”). From terminal A (FIG. 4B), the method 400 proceeds to block 416, where the user interface engine 208 collects environmental information regarding ambient temperature, humidity, and/or pollution levels to which the subject 90 is exposed. In some embodiments, the user interface engine 208 may present a questionnaire that directly collects the environmental information from the subject 90. In some embodiments, the user interface engine 208 may present a questionnaire that collects information from the subject 90, such as a location, that can be used to look up the environmental information from publicly available information sources for the location.

At block 418, the mobile computing device 102 transmits the at least one pH value, the image of the subject, the environmental information, and the indication of the skin condition to a server computing system 104. In some embodiments, the mobile computing device 102 may transmit the pH measurements obtained from the electronic measurement devices 106 instead of the pH values determined by the mobile computing device 102, such that the processing of the pH measurements may be performed by the server computing system 104 instead of the mobile computing device 102.

At block 420, a product recommendation engine 212 of the server computing system 104 determines at least one recommended skincare product based on the information received from the mobile computing device 102. In some embodiments, the product recommendation engine 212 may retrieve at least one recommended skincare product from the product data store 214 that is specifically associated with regulating the detected pH in order to treat the skin condition subject to the specified environmental conditions. In some embodiments, a machine learning model such as a recommender system may be used to determine the at least one recommended skincare product.

At block 422, a visualization generation engine 210 of the server computing system 104 generates a visualization of the subject 90 based on the information received from the mobile computing device 102. In some embodiments, a prediction of an effect that the recommended skincare product will have on the skin condition subject to the specified environmental conditions will be generated. For example, the visualization generation engine 210 may determine that a given skincare product will adjust a skin pH from a problematic value detected by the system to a normal value, and may then use computer image generation techniques generate a visualization that depicts the subject 90 as having less evidence of the skin condition.

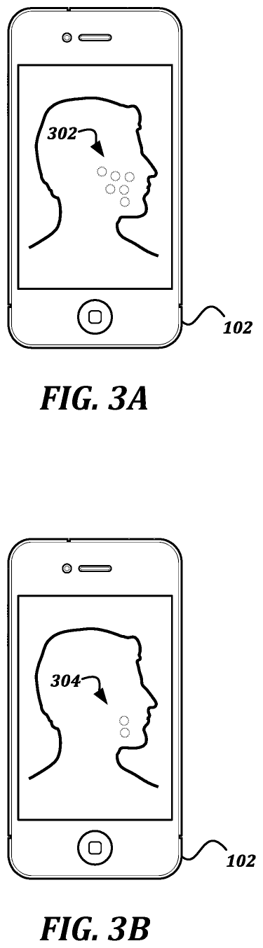

FIGS. 3A and 3B are illustrations showing an example of a first image taken of a subject and a predictive visualization generated of the subject according to various aspects of the present disclosure. In FIG. 3A, an image captured of the subject is presented, showing multiple acne lesions. In FIG. 3B, a predictive visualization of the subject is presented, showing a predicted reduction in acne lesions after recommended use of a recommended skincare product.

Returning to FIG. 4B, at block 424, the server computing system 104 stores the information received from the mobile computing device 102 in a results data store 216. In some embodiments, this information may later be associated with feedback received from the subject 90, and/or to improve recommendations provided by the product recommendation engine 212. At block 426, the server computing system 104 transmits the at least one recommended skincare product and/or the visualization of the subject 90 to the mobile computing device 102.

The method 400 then proceeds to another continuation terminal (“terminal B”). From terminal B (FIG. 4C), the method 400 proceeds to block 428, where the user interface engine 208 presents the at least one recommended skincare product and/or the visualization of the subject 90. At block 430, after a recommended treatment regimen, an updated image of the subject 90, environmental information, and one or more pH values are captured and/or determined by the mobile computing device 102. In some embodiments, similar techniques as those described in blocks 406-416 may be used to again capture this information. At block 432, the mobile computing device 102 transmits the updated information to the server computing system 104. At block 434, the server computing system 104 stores the updated information in the results data store 216. In some embodiments, the updated information is associated with the original information stored at block 424.

At block 436, the server computing system 104 uses the updated information to improve future skincare product recommendations, and at block 438, the server computing system 104 uses the updated information to improve future visualization generations. For example, the server computing system 104 may compare the updated image of the subject 90 to the original image collected of the subject, and may use computer vision techniques such as convolutional neural networks to detect skin regions that exhibit signs of the skin condition. The continued presence, absence, or reduction of signs of the skin condition can be used to determine the effectiveness of the recommended skincare product, and this effectiveness can then be used to either increase or reduce the likelihood that the skincare product will be recommended in the future for subjects that share traits (such as similar skin pH or similar environmental factors) with the subject 90. The differences between the updated image and the original image can also be used to generate similar differences in visualizations for other subjects.

The method 400 then proceeds to an end block and terminates.

FIG. 5 is a flowchart that illustrates a non-limiting example embodiment of a method of determining pH values based on measurements generated by an electronic measurement device according to various aspects of the present disclosure. The method 500 is an example of a method for determining pH values suitable for use at block 414 of the method 400 described above. In the method 500, an electronic measurement device 106 transmits measurements to a mobile computing device 102, and the mobile computing device 102 determines at least one pH value based on the measurements.

From a start block, the method 500 proceeds to block 502, where a pH measurement electrode of an electronic measurement device 106 is used to measure electrical potentials at a skin contact point. In some embodiments, a measurement surface that includes at least a sensor electrode is placed against the skin contact point. In some embodiments, a conductive fluid, such as deionized water, may be used to wet the skin contact point before the measurement is taken. In some embodiments, particularly embodiments wherein the electronic measurement device 106 is a wearable device that is held in contact with the skin contact point for long periods of time, sweat or other fluids generated by the skin and trapped under the electronic measurement device 106 may be sufficient to hydrate the sensor electrode. In some embodiments, the electronic measurement device 106 may include a heater or other means for stimulating sweat production at the skin contact point.

The electronic measurement device 106 may generate a single measurement of electrical potential, or may generate multiple measurements of electrical potential while the sensor electrode is in contact with the skin contact point. The electrical potential sensed by the sensor electrode may be a potential between the sensor electrode and a reference electrode, and may be correlated to the pH of the skin. Typically, the method 500 may be used for a single skin contact point, and information for other skin contact points may be obtained by performing the method 500 multiple times. In some embodiments, the method 500 may collect information from more than one skin contact point.

At block 504, a temperature sensor 706 of the electronic measurement device 106 is used to measure temperatures at the skin contact point. Each temperature measurement is paired with an electrical potential measurement. The temperature measurement may be used to correct temperature-based biases in the electrical potential measurements.

At block 506, one or more electrical potential measurements and one or more temperature measurements are transmitted to a mobile computing device 102. The measurements may be transmitted to the mobile computing device 102 using any suitable technique. For example, the electronic measurement device 106 may transmit the measurements via a wireless network (including but not limited to Bluetooth, Wi-Fi, Zigbee, or NFC) or a wired network (including but not limited to USB or FireWire). As another example, the electronic measurement device 106 may record the measurements on a removable computer-readable medium, such as a flash memory, and the computer-readable medium may be transferred to the mobile computing device 102. As yet another example, a display device on the electronic measurement device 106 may present the measurements, and the measurements may be input into the mobile computing device 102 via a user interface, or may be captured by a camera of the mobile computing device 102.

At block 508, a pH determination engine 206 of the mobile computing device 102 determines proposed pH values based on the electrical potential measurements and the temperature measurements. In some embodiments, the proposed pH values may be determined by consulting one or more look-up tables or performing other calculations to determine a preliminary pH value associated with the electrical potential measurement. This preliminary pH value may then be adjusted based on the associated temperature measurement to determine the proposed pH value. In some embodiments, a look-up table or calculation may be performed that directly determines the proposed pH value based on the combination of the electrical potential measurement and the temperature measurement. A proposed pH value may be determined for each of the electrical potential measurements.

At block 510, the pH determination engine 206 extracts proposed pH values within tolerance limits to reject unreliable data points. For example, if the proposed pH values fall outside of a predetermined range of plausible pH values for human skin, the proposed pH values may be rejected. This may allow the pH determination engine 206 to ignore proposed pH values that were measured without the electronic measurement device 106 being properly in contact with the skin contact point, or proposed pH values that were generated in a faulty manner for some other reason.

At block 512, the pH determination engine 206 determines a pH value based on the proposed pH values. In some embodiments, the pH determination engine 206 may determine an average (a mean, a median, or a mode) of the proposed pH values in order to determine the pH value. The method 500 then proceeds to an end block and terminates.

FIG. 6 is a flowchart that illustrates a non-limiting example embodiment of a method of determining pH values based on measurements generated by an electronic measurement device using machine learning according to various aspects of the present disclosure. The method 600 is another example of a method for determining pH values suitable for use at block 414 of the method 400 described above. In the method 600, a machine learning model is generated that can be used to determine pH values based on measurements obtained by an electronic measurement device 106, and the model is then used by a mobile computing device 102 to process such measurements.

From a start block, the method 600 proceeds to block 602, where an electronic measurement device 106 collects measurements of at least electrical potentials and temperatures of samples having known pH values in varying environments. As in block 502 of method 500, the measurement surface may be placed against a sample having a known pH value in the presence of a conductive fluid, and the electronic measurement device 106 may generate one or more measurements of electrical potential and temperature. In some embodiments, multiple different samples having different known pH values may be used, and measurements may be obtained at different temperatures, different hydration conditions, and so on, in order to produce a variety of training data.

At block 604, a machine learning model is trained using the measurements of the samples having known pH values. The measurements may be tagged with the known pH values in order to create a set of supervised training data, and a machine learning model (including but not limited to an artificial neural network) may be trained with the training data using any suitable technique, including but not limited to gradient descent. The resulting machine learning model will accept an electrical potential and a temperature as an input, and will output a potential pH value. In some embodiments, the training data and machine learning model may use series of measurements as input in order to avoid errors introduced by processing only a single measurement (that could be erroneous).

At block 606, an electronic measurement device 106 collects measurements of at least electrical potentials and temperatures of a skin area of a subject having an unknown pH value. These actions are also similar to those described in block 502, and so are not described in detail here for the sake of brevity.

At block 608, a pH determination engine 206 of the mobile computing device 102 receives the measurements and discards measurements outside of tolerance limits to reject unreliable data points. For example, the pH determination engine 206 may discard electrical potential measurements that are outside of a plausible range for the electrical potential of human skin. This may allow the pH determination engine 206 to avoid processing measurements of air or other measurements taken when the electronic measurement device 106 is not properly in contact with the skin area.

At block 610, the pH determination engine 206 determines the unknown pH value using the machine learning model. In some embodiments, the measurements are provided to the machine learning model using a technique similar to the technique used to provide measurements during the training actions in block 604, such as individually or as a series. The output of the machine learning model is at least one pH value determined in response to the measurements provided as input.

The method 600 then proceeds to an end block and terminates.

The specific actions described in method 600 are examples only, and in some embodiments, different actions might be taken. For example, in some embodiments, the machine learning model may be stored on the electronic measurement device 106 once trained by a separate computing device, and the electronic measurement device 106 may itself determine the pH values using the machine learning model. As another example, in some embodiments, the measurements may be processed to remove unreliable data points from the training data as well. As yet another example, in some embodiments, instead of discarding unreliable data points from the measurements, all of the measurements may be provided to the machine learning model, and pH values that are outside of a range of plausible pH values for human skin may be discarded.

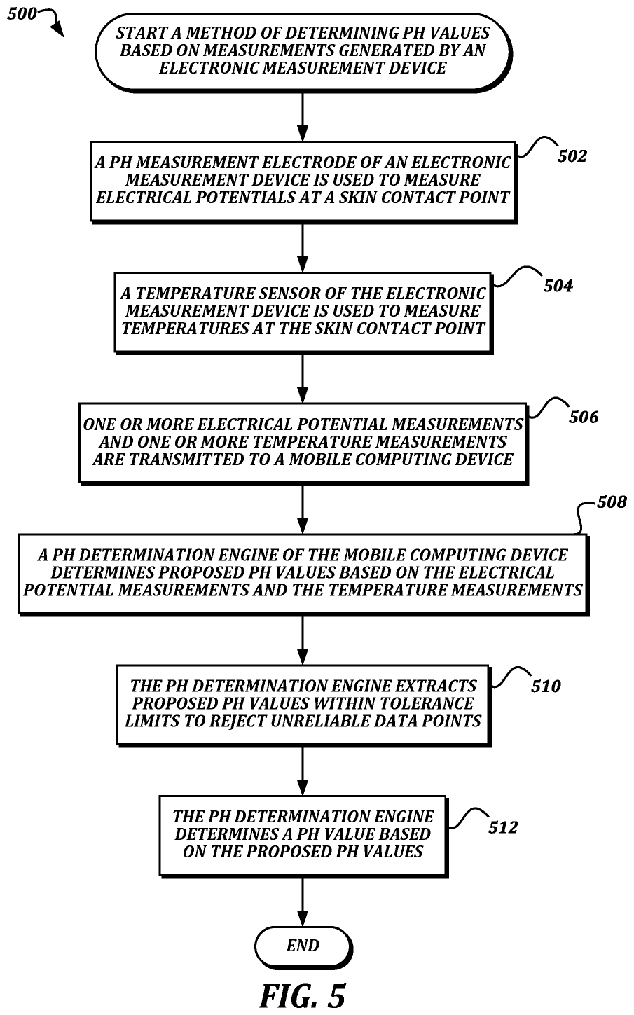

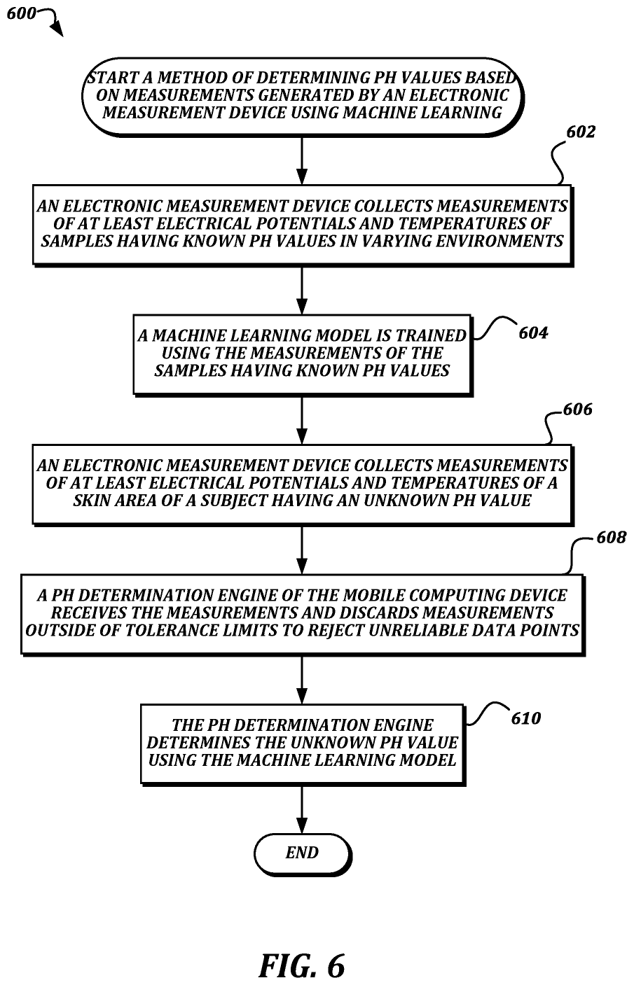

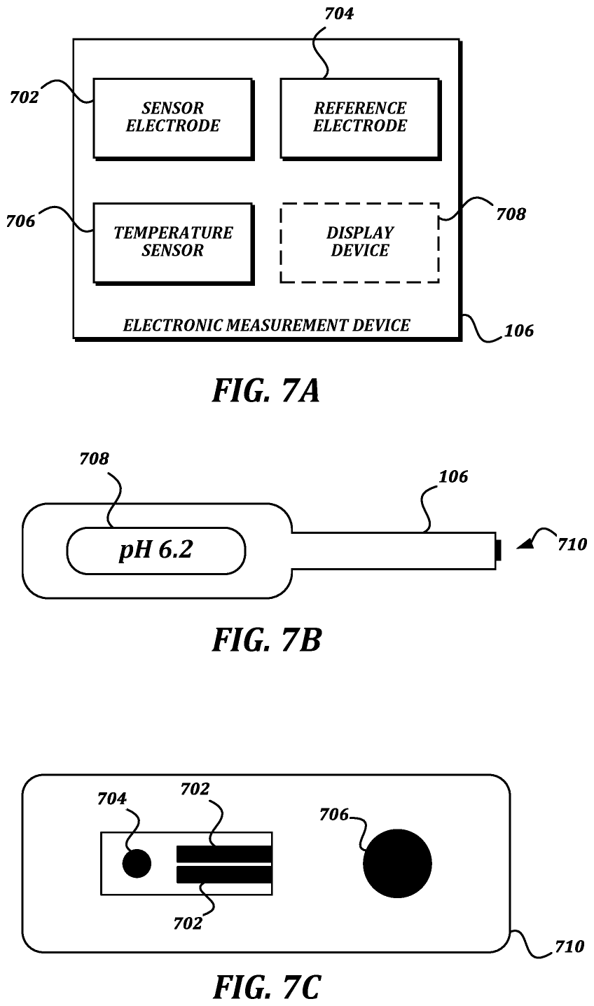

FIG. 7A is a block diagram that illustrates example components included within a non-limiting example embodiment of an electronic measurement device 106 according to various aspects of the present disclosure. As shown, the electronic measurement device 106 includes a sensor electrode 702, a reference electrode 704, a temperature sensor 706, and an optional display device 708.

In some embodiments, the sensor electrode 702 and the reference electrode 704 are configured to detect an electrical potential when the electrodes 702, 704 are placed against a surface to be analyzed (such as a skin location). The sensor electrode 702 and reference electrode 704 may be included within a sensor device. Any type of sensor device having electrodes capable of measuring electrical potentials in human skin may be used, including but not limited to an ISFET sensor, a glass electrode sensor, or an antimony pH sensor.

In some embodiments, the temperature sensor 706 is configured to detect a temperature of a surface contacted by the temperature sensor 706. In some embodiments, the temperature sensor 706 is positioned such that it will be in contact with a surface being analyzed by the electrodes 702, 704, such that a temperature of the analyzed surface can be determined. Any type of temperature sensor may be used, including but not limited to a negative temperature coefficient (NTC) thermistor, a resistance temperature detector (RTD), a thermocouple, or a semiconductor-based sensor.

In some embodiments, an optional display device 708 may be included. The display device 708 may be an LCD display, an LED display, an OLED display, or any other type of device suitable for presenting information to a user. The display device 708 may be configured to present pH values detected by the electronic measurement device 106, and/or may be configured to present instructions or guidance to a user for obtaining measurements using the electronic measurement device 106. The display device 708 is illustrated as optional because, in some embodiments, the display device 708 may not be included. This may be particularly likely for electronic measurement devices 106 that are configured to be wearable devices.

In some embodiments, the electronic measurement device 106 may also include additional components other than those illustrated in FIG. 7A, including but not limited to a battery (or other power source), and a communication interface. These components have not been illustrated for the sake of brevity.

FIG. 7B illustrates a non-limiting example embodiment of an electronic measurement device 106 according to various aspects of the present disclosure. The electronic measurement device 106 illustrated in FIG. 7B is configured as a probe device, which would be temporarily placed against a skin location to obtain a measurement of the skin location. As shown, the electronic measurement device 106 includes a display device 708 that is displaying a measurement obtained by the electronic measurement device 106. In use, a measurement surface 710 of the electronic measurement device 106 is placed against the skin location to be measured.

FIG. 7C illustrates a non-limiting example embodiment of a measurement surface 710 of an electronic measurement device 106 according to various aspects of the present disclosure. As shown, the measurement surface 710 includes sensor electrodes 702 and a reference electrode 704. The potential measured between the sensor electrodes 702 and the reference electrode 704 is correlated to a pH of the surface with which the electrodes are in contact. The measurement surface 710 also includes a temperature sensor 706, which is arranged to be in contact with the same surface as the electrodes 702, 704. Similar measurement surfaces 710 may be used with electronic measurement devices 106 of different form factors. For example, a similar measurement surface 710 may be used whether the electronic measurement device 106 is a temporary-use probe as illustrated in FIG. 7B or a wearable device. In some embodiments, instead of providing the electrodes on a measurement surface 710, a microfluidic system may be provided that samples fluids from the skin and provides the fluids to the sensor electrode 702, reference electrode 704, and temperature sensor 706 at a location other than the skin contact point.

While illustrative embodiments have been illustrated and described, it will be appreciated that various changes can be made therein without departing from the spirit and scope of the invention.

The embodiments of the invention in which an exclusive property or privilege is claimed are defined as follows:

1. A system for generating predictive visualizations of a result of a skin treatment regimen, the system comprising:
a server computing system comprising one or more computing devices;
at least one electronic measurement device configured to produce measurements of a skin area of a subject on which the electronic measurement device is placed; and
a mobile computing device comprising:
at least one processor;
a display device;
a camera; and
a non-transitory computer-readable medium having computer-executable instructions stored thereon that, in response to execution by the at least one processor, cause the mobile computing device to:
capture an image of an area of interest of the skin of the subject;
receive measurements from the electronic measurement device;
determine a pH value representing a pH of the skin area of the subject by analyzing the measurements produced by the electronic measurement device; and
transmit the pH value and the image of the area of interest to the server computing system;
wherein the server computing system is configured to:
determine a skincare product recommendation based on the pH value;
generate a visualization indicating a result of applying the skincare product to the area of interest based on the pH value, the image of the area of interest, and the skincare product; and
transmit the visualization to the mobile computing device;
wherein the instructions further cause the mobile computing device to:
receive and present the visualization to the subject.

2. The system of claim 1, wherein the instructions further cause the mobile computing device to:
collect environmental information regarding at least one of ambient temperature, humidity, and pollution levels; and
transmit the environmental information to the server computing system;
wherein the determination of the skincare product recommendation is further based on the environmental information.

3. The system of claim 1, wherein the instructions further cause the mobile computing device to:
collect information regarding a medical condition experienced by the subject; and
transmit the information regarding the medical condition to the server computing system;
wherein the determination of the skincare product recommendation is further based on the information regarding the medical condition.

4. The system of claim 3, wherein the medical condition is selected from a group consisting of eczema, atopic dermatitis, and acne vulgaris.

5. The system of claim 3, wherein the visualization illustrates a predicted improvement in the medical condition based on a predicted effect of application of the skincare product.

6. The system of claim 1, wherein the visualization illustrates a predicted pH trend caused by application of the skincare product.

7. The system of claim 1, wherein receiving measurements from the electronic measurement device includes receiving measurements associated with multiple skin regions, and wherein the recommendation includes at least two skincare products recommended for use on different skin regions.

8. The system of claim 7, wherein the instructions further cause the mobile device to present instructions for placement of the electronic measurement device with respect to each skin region.

9. The system of claim 1, wherein the at least one electronic measurement device includes an ISFET sensor, a glass electrode sensor, or an antimony pH sensor.

10. A method for treating a patient with a skin condition associated with high skin pH, the method comprising:
determining a skin pH of skin of the patient by:
obtaining at least one measurement using at least one electronic measurement device from the skin of the patient, wherein the at least one measurement includes an electrical potential of the skin; and
using a mobile computing device to determine the skin pH based on the electrical potential of the skin;
determining a recommended skincare product to treat the skin condition based on the skin pH; and
topically administering the recommended skincare product to the skin.

11. The method of claim 10, wherein the skin condition is selected from a group consisting of atopic dermatitis, eczema, and acne vulgaris.

12. The method of claim 10, wherein the recommended skincare product is selected from a group consisting of a syndet, a soap-based cleanser, an alpha-hydroxy acid, and an acidic electrolyte water.

13. The method of claim 10, wherein the at least one electrical measurement device includes at least one of an ISFET sensor, a glass electrode sensor, or an antimony pH sensor.

14. The method of claim 10, further comprising collecting environmental information regarding at least one of ambient temperature, humidity, and pollution levels, wherein the determination of the recommended skincare product is further based on the environmental information.

15. A method of generating a rendering of a face image to indicate a result of a skin treatment regimen, the method comprising:
receiving, by a mobile computing device, a measurement of a skin area of a subject, wherein the measurement is associated with a pH of the skin area;
capturing, by the mobile computing device, an image of a face of the subject;
determining, by the mobile computing device, a pH value based on the measurement;
determining, by the mobile computing device, a recommended skincare product based on the pH value;
determining, by the mobile computing device, a predicted face image based on the pH value, the recommended skincare product, and the second image; and
presenting, by the mobile computing device, the predicted face image to the subject.

16. The method of claim 15, further comprising collecting, by the mobile computing device, environmental information regarding at least one of ambient temperature, humidity, and pollution levels;
wherein the determination of the recommended skincare product and the determination of the predicted face image are both further based on the environmental information.

17. The method of claim 15, further comprising receiving, by the mobile computing device, an indication of a medical condition experienced by the subject;
wherein determining the predicted face image is further based on the indication of the medical condition, and the predicted face image indicates an effect of the recommended skincare product on the medical condition.

18. The method of claim 17, wherein the medical condition is selected from a group consisting of eczema, atopic dermatitis, and acne vulgaris.
